(19) United States
(12) Patent Application Publication
(10) Pub. No.: US 2026/0166438 A1
(43) Pub. Date: Jun. 18, 2026

(54) Systems And Methods for Gameplay Recommendations

(71) Applicant: ATI Technologies ULC, Markham (CA)

(72) Inventors: Joseph Michael Gravenor, Toronto (CA); Ilia Blank, Concord (CA); Panagiotis Vagiakos, Toronto (CA); Wei Liang, Markham (CA); Le Zhang, Markham (CA); Sumalata Hiremath, Richmond Hill (CA); Shanmukha Sai Vignesh Edithal, Toronto (CA)

(21) Appl. No.: 18/982,692

(22) Filed: Dec. 16, 2024

(51) Int. Cl. A63F 13/67; A63F 13/213; G06F 3/01; G06V 40/16; G16H 20/70

(52) U.S. Cl. CPC A63F 13/67; A63F 13/213; G06F 3/011; G16H 20/70; G06F 2203/011; G06V 40/174

(57) ABSTRACT

Systems and methods for generating gameplay recommendations are described. A recommendation system detects a user device login for a gaming application and collects video data and biometric data from the user device. The video data includes gameplay video data and camera feed from a camera associated with the user device. The biometric data is collected from one or more biometric devices connected to the user device. The system detects termination of an application session and computes emotional state data for a user of the user device based on the camera feed and the biometric data. The system also detects in-game events based on the gameplay video data. Based on the detected in-game events and respective outcomes correlated with the computed emotional state data, the recommendation system generates gameplay recommendation data.

### BACKGROUND

### Description of the Related Art

Gaming applications allow users to access a variety of games hosted on various platforms, such as consoles, PCs, mobile devices, or through cloud-based services over a network like the Internet. Users log into their accounts, choose a game from the list available to them, and initiate gameplay using their device of choice. When a game is selected, the platform or device processes the game session, rendering the game for the user. If the user exits the session, the gameplay is ended, and a brief session summary indicating gameplay performance is provided to the user's device. This summary can include key performance metrics, such as total points scored, kills, deaths, levels completed, achievements unlocked, time played, and so on. However, players often struggle to determine why certain events occurred or why they did not perform better. For example, some players may perform best when calm, and other players may perform better when agitated. Furthermore, a way of receiving personalized insights and assistance during gameplay, is traditionally a service that is obtained from external sources and therefore can be inconvenient and/or expensive.

In view of the above, improved systems and methods for gameplay recommendations are needed.

### BRIEF DESCRIPTION OF THE DRAWINGS

The advantages of the methods and mechanisms described herein may be better understood by referring to the following description in conjunction with the accompanying drawings, in which:

FIG. 1 is a block diagram illustrating one network implementation of a recommendation system.

FIG. 2 is a block diagram illustrating one implementation of various components of the recommendation system.

FIG. 3 is a block diagram illustrating generation of a session file for a user device.

FIG. 4 is a block diagram illustrating generation of recommendations for a user interacting with a gaming application session.

FIG. 5 is a block diagram illustrating generation of gameplay recommendations.

FIG. 6 illustrates a method for generating gameplay recommendations.

FIG. 7 illustrates another method for generating gameplay recommendations.

### DETAILED DESCRIPTION OF IMPLEMENTATIONS

In the following description, numerous specific details are set forth to provide a thorough understanding of the methods and mechanisms presented herein. However, one of ordinary skill in the art should recognize that the various implementations may be practiced without these specific details. In some instances, well-known structures, components, signals, computer program instructions, and techniques have not been shown in detail to avoid obscuring the approaches described herein. It will be appreciated that for simplicity and clarity of illustration, elements shown in the figures have not necessarily been drawn to scale. For example, the dimensions of some of the elements may be exaggerated relative to other elements.

Systems, apparatuses, and methods for gameplay recommendations are described. A recommendation system uses Facial Emotion Recognition (FER) technology and heart rate monitoring to assess a gamer's emotional state during gameplay. It analyzes video frames from the webcam of the gamer's user device, to identify facial expressions. The system further monitors the player's biometric data through connected biometric devices, such as a smartwatch. At the same time, the recommendation system utilizes a separate machine-learning model to identify important in-game events. At the end of the gaming session, the system consolidates the emotional state data and the in-game event data and presents this data to the user device. To further enhance the user experience, the system can utilize other machine learning models to analyze the compiled data and provide personalized advice and recommendations on how to augment the user's emotional state. The system further provides the user with game recommendations to positively impact their emotional state.

In an implementation, an “application session” as described herein refers to a period of time during which a user interacts with a software application or program. During such a session, a user may engage with the application performing various tasks or actions, e.g., via a user interface. According to the implementation, an application session can begin when the user starts the application or opens a particular instance of the application on a user device. During an application session, users can perform different actions such as inputting data, navigating through different screens or sections, executing commands, modifying settings, and accessing various features or functions provided by the application. The interaction of the user with the application can also generate session-related data, such as user preferences, temporary data, or the application's state. In an implementation, session duration can vary depending on the nature of the application and the user's objectives. For example, a session on a productivity application or game might last for hours, other sessions may last only minutes.

In one implementation, an application session can include gameplay session. In order to participate in the gameplay, a player logs in to a gaming application, using their personal computers, gaming consoles, or mobile devices. For example, the user can join a virtual environment or game world where they can interact with other players who are also connected to the same server. The gameplay session for that player begins when the player logs into the game and ends when they log out or disconnect from the server. In the description that follows, the terms “gameplay session” and “session” are used interchangeably.

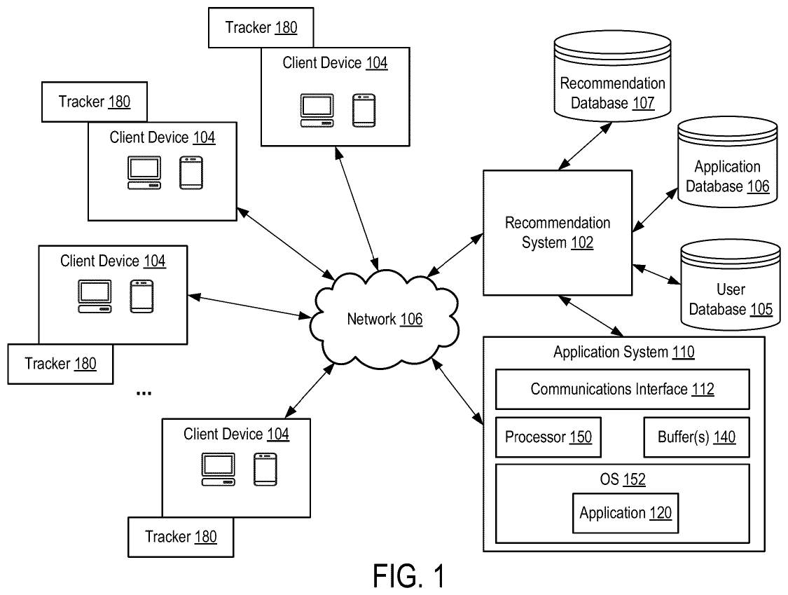

FIG. 1 is a block diagram illustrating a network implementation of a recommendation system. As shown, a recommendation system 102 (alternatively referred to as recommendation system 102) is connected to an application system 110, which in turn is connected to a plurality of client devices 104 (hereinafter also referred to as user devices 104) over a network 106. In an implementation, the application system 110 is configured to provide the core application functionality that allows users using client devices 104 to engage with application(s) over the network 106. In an example, for cloud-based versions, parts of the application system 110 can be integrated into the application 120. The communications interface 112 includes various components that facilitate communication across networks, like network(s) 106. This interface handles tasks such as sending and receiving user inputs from client devices 104, as well as transmitting application data.

As described herein, in one implementation, the application 120 can be any type of software capable of executing application sessions on the client device 104. Examples of applications 106 can include mobile applications, computer programs, console applications, cloud-based game streaming software, web browsers, game applications, local or client-based applications, social applications, system or native applications, and other types of software. In one implementation, when executing applications, a client device 104 is configured to receive input data from application system 110, execute application session(s), and send resulting data back to application system 110. The client device 104 is further configured to retrieve application-related data from memory or local storage, and receive execution instructions from application system 110.

Client devices 104 can include a range of devices, such as smartphones, laptops, tablet computers, desktop computers, wearable technology, game consoles, virtual reality systems (e.g., VR headsets, computers, controllers, or other components), streaming devices, smart-home devices with intelligent personal assistants, or any other type of device capable of supporting various applications, e.g., gameplay applications.

In an implementation, the application system 110 includes various buffers 140, and at least one processor 150 executing an operating system 152 which hosts the application 120. In an implementation, the processor 150 processes connection requests received from a given client device 104, e.g., requesting activation of an application session of an application. The request is received by the application system 110 over the network 106, e.g., Internet. In one example, the client device 104 can request the application system 110 to access a cloud gaming site or a web based gaming application. Responsive to receiving the request, the processor 150 determines whether the client device 104 is authorized to access the requested application. In an example, client devices registered with the application system 110 may have access to one or more applications, stored in the application database 106. In some implementations, the processor 150 processes requests to access one or more applications and responds to those requests, for example, by facilitating authorization of login credentials for the client device 104. For example, for cloud gaming applications, the client device 104 is provided access to one or more user interfaces, so that a user can engage with an application session. In one or more implementations, the application system 110 and the client device 104 are part of the same computing device. Such implementations are contemplated.

The processor 150 is further configured to access user data stored in the user database 105 in order to process incoming requests. The user data, in one example, comprises of information such as but not limiting to, username, password, registration date, email address, phone number, subscription ID, application usage history, social graph information, application ownership, preferences, and other settings. In an implementation, if the client device 104 is unregistered, the processor 150 provides an option to register. A user of the client device 104 can enter required registration information and register the client device 104. In an implementation, identification of whether a client device is a registered or unregistered device may be made at least based on user data stored at user database 105. Further, once the application session is active on the client device 104, data generated as a result of a user engaging with the application session is received by the application system 110 and stored in one or more buffer(s) 140. This is done per application session per user.

The buffer(s) 140 can include one or more storage devices used to store or temporarily hold data generated during execution of an application session, before it is transmitted over the network 106, e.g., to the recommendation system 102. In an implementation, data set for transmission can be stored in any given format, e.g., using multiple queues simultaneously, where data from a single application session can be stored in one or more queues, and data from different sessions can either be stored in the same queue or in separate queues. In one example, the buffer(s) 140 can be used to temporarily store video frames or images, such as video frames recorded during various application sessions etc. This video data can be accessed from the buffer(s) 140 by the recommendation system 102.

In one implementation, user data further includes, without limitation, biometric data collected from one or more biometric trackers 180, such as a fitness tracker or heart rate monitor worn by a player using a user device 104. The user data further includes video frames recorded during an application session, as well as video images of the player during the application session, e.g., captured via a webcam internal or otherwise connected to the user device 104. In an implementation, the video frames recorded during an application session are used by the recommendation system 102 to identify application events. Further, facial expressions identified from webcam video data along with biometric data is used to compute emotional state data of the user. A wide range of other data types and methods for capturing such data can also be utilized in different implementations.

In one or more implementations, recommendation system 102 is configured to generate recommendations for users of the user devices 104. In one such implementation, the recommendation system 102 is configured to save user data for each user to an internal storage or cloud storage, thereby creating a personalized profile for each user. A given profile enables the recommendation system 102 to recommend applications for a user based on user's emotional state data. There profiles are hereinafter referred to as “session files.” In an implementation, for each user device 104, a session file is generated (and updated) whenever the user device 104 completes a given session of a given application.

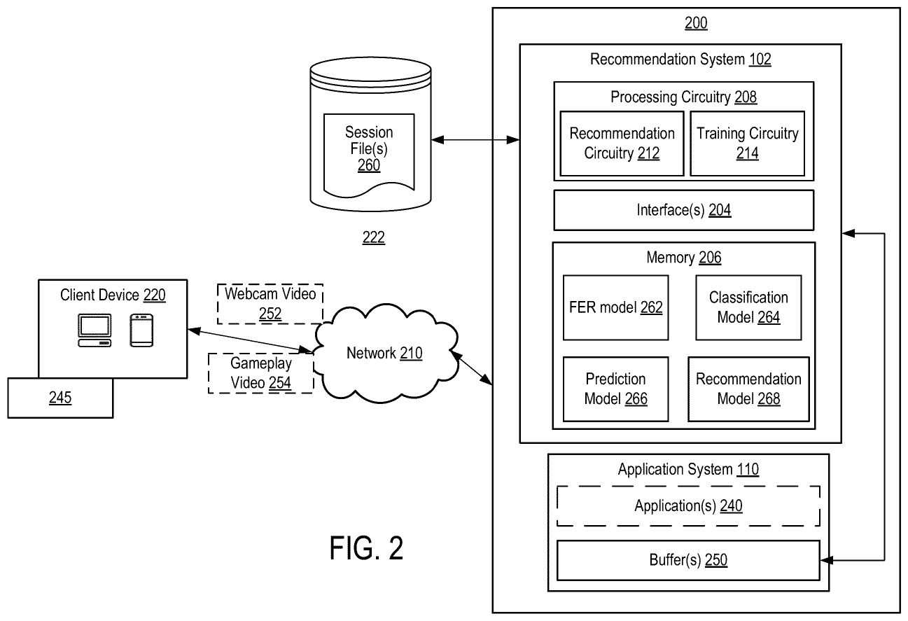

In one implementation, for gaming applications executing on a user device 104, the recommendation system 102 is configured to track in-game events as well as a user's emotional state data (described in detail using FIGS. 2-6) during a gaming session on the user device 104. After the gaming session terminates, the recommendation system 102 generates for presentation, e.g., on a graphical user interface (GUI) of the user device 104, a visual representation of the user's emotional state data and corresponding recorded in-game events. Further, the recommendation system 102 also generates recommendation data indicative of a correlation between the user's gameplay performance and their emotional state data. In other implementations, the recommendation system 102 is further configured to generate recommendation data that can help a user to achieve a certain mood, e.g., as identified using the emotional state data. For example, if improved gameplay is associated with a “happy” mood, then recommendation data can indicate what events or circumstances are correlated with a happy emotional state. In such implementations, the system 102 integrates user data analysis, including analyzing real-time emotional state data of the user using inputs such as facial expressions, audio data, or interaction patterns. The identified mood is processed using one or more machine learning models, e.g., to correlate user's current emotional state with a curated database of applications. In some examples, these applications are categorized and tagged according to mood-appropriate functionalities, such as relaxation, productivity, entertainment, or social interaction. The system 102 optimizes user experience by dynamically updating recommendations as the user's mood fluctuates, enhancing engagement and user satisfaction.

In one implementation, to identify in-game events, the recommendation system is configured to process video frames stored in the buffer(s) 140, e.g., using a machine-learning model. In an example, when an event is detected, the recommendation system 102 adds a type of the detected event along with a timestamp of occurrence of the event to a session file (e.g., JSON file). In an implementation, the recommendation system 102 further processes the emotional state data, generated based on facial expressions and biometric data (e.g., heart rate data), through a neural network. The recommendation system 102 analyzes this data to detect how the emotional state data influences relevant gaming events. Based on this analysis, the recommendation system 102 is configured to generate recommendations that indicate how different sets of emotional state data can affect a user's gameplay. Further, the recommendations include one or more alternate gaming applications to for the user selected based on detected emotional state data of the user. Once the gaming application session terminates, the recommendation system 102 is configured to present recommendation data to the user device 104. These and other implementations are described in the text that follows.

In one or more implementations, the recommendation system 102 includes, or is otherwise connected to, one or more databases. As shown, these databases at least include user database 105, application database 106, and recommendation database 107. In one example, data corresponding to each user device 104, including but not limiting to, video data, biometric data, session files, and emotional state data can be stored using the user database 105. The user database 105 can further store login information of each user for different applications, as well as permissions settings corresponding to each user device 104. These permission settings can govern what type of data can be retrieved by the recommendation system 102 from the user device 104 during execution of a given application session. The application database 106 is configured to store various applications, such as gaming applications, such that client devices 104 registered with the recommendation system 102 can have access to one or more applications, stored in the application database 106. Further, recommendation database 107 can store recommendation data generated by the recommendation system 102. Other implementations are possible and are contemplated.

The implementations described herein aims to offer users, such as gamers, personalized insights, and assistance during gameplay. Traditionally, such assistance is obtained from external sources and can be inconvenient and/or expensive. The methods and systems described are able to leverage existing user equipment, e.g., webcam and biometric devices, and utilizes machine learning algorithms to detect in-game events. This approach enables seamless provision of sending recommendation data to the user devices, eliminating the need for human monitoring during gameplay.

Turning now to FIG. 2, a block diagram illustrating various components of the recommendation system 102 is described. It is noted that the FIG. 2 describes the recommendation system 102 as configured to generate recommendation data for a user engaging with a gaming application, however, the methods described herein can similarly be applied to other types of applications. Such implementations are contemplated.

In one or more implementations, the recommendation system 102 (or simply “system 102”) uses Facial Emotion Recognition (FER) and sensor data monitoring (e.g., heart rate) to assess a user's emotional state during gameplay. The system 102 is configured to analyze video frames recorded during a gameplay session, e.g., from a webcam integrated in a user device, to identify facial expressions of the user interacting with the gameplay session. The system 102 further monitors the user's heart rate data. This data can be retrieved through various biometric devices (e.g., smartwatch, fitness tracker, or other smart wearables). The system 102 further utilizes a machine-learning model to identify important in-game events. At the end of the gameplay session, the system 102 correlates the emotional state data and the identified in-game events to generate recommendation data for improving the user's gameplay. The system 102 further uses various machine learning models (as described in the text that follows) to analyze gameplay data and provide personalized advice and recommendations to improve the user's emotional state, e.g., different moods identified based on the user's emotional state. Furthermore, the system 102 is configured to generate recommendation data to alter a currently identified emotional state of the user. The recommendation data is presented on a graphical user interface (GUI) of the user device at the end of every session.

In the implementation shown in the figure, the recommendation system 102 comprises one or more interface(s) 204, a memory 206, and processing circuitry 208. In an implementation, the one or more interface(s) 204 are configured to display data generated as a result of the processing circuitry 208 executing one or more programming instructions stored in the memory 206. In one implementation, the processing circuitry 208 includes multiple cores configured to execute instructions. In some implementations, the processing circuitry 208 further includes circuitry configured to perform parallel processing. In some implementation, the processing circuitry 208 is a system on a chip (SOC) including multiple hardware components (e.g., CPU, GPU, memory controller, etc.). Multiple such implementations are possible and are contemplated. For instance, as shown, the processing circuitry 208 includes circuitries including recommendation circuitry 212, and training circuitry 214, in order to perform one or more functions described herein. The system 102 is connected to one or more user devices 220, through a network 210, such as the Internet. In one or more implementations, circuits and/or circuitries described herein can be implemented using field programmable gate arrays (FPGAs).

The system 102 further includes an application system 110 which is configured to host various gaming applications 240 as requested by one or more user devices 220. In the implementation shown herein, the application system 110 and recommendation system 102 are part of the same computing unit, e.g., a game server 200. However, in other implementations, the recommendation system 102 and the application system 110 can be standalone computing systems (as described in FIG. 1) or operate as separate instances of the same computing system. Further, in some implementations, the application system 110 and a user device 220 can also be part of the same computing system or environment. Such implementations are contemplated.

In operation, the application system 110 manages permissions corresponding to each user device 220. This includes managing permissions to access data from one or more biometric sensors 245 associated with each user device 220. These biometric sensors 245 can include fitness trackers, smartwatches, heart rate sensors, or other smart wearables. In one implementation, the application system 110 is configured to present options to a user device 220 to manage permissions, when the user device 220 first signs up for a gaming application 240 through the application system 110. For instance, when a user device 220 signs up to access a gaming application 240, the application system 110 queries the user device 220 for permissions relating to use of various data. These permissions can at least include permission to store and access biometric data and webcam data generated during each session of the gaming application 240. The selected permission settings for each user device 220 is stored in a user database 222. Once these permissions are enabled at the user device 220 end, the recommendation engine 212 can begin recording data for each application session and provide recommendation data to the user device 220 at the end of each session.

In one or more implementations, the data recorded includes video data from a webcam (or other integrated imaging device) corresponding to the user device 222 and biometric data gathered from at least one biometric sensor 245 associated with the user device 222. In one example, the biometric data is obtained by accessing an application programming interface (API) of a biometric sensor 245, such as a smartwatch. In case no biometric sensors 245 are available and/or the data from biometric sensors 245 is not otherwise accessible, only webcam data may be recorded.

During gameplay, i.e., when a session of a gaming application 240 is hosted by the application system 110 for a user device 220, the application system 110 accesses video frames from a webcam of the user device 220 (webcam video 252). The application system 110 further accesses video frames corresponding to the gameplay session, e.g., video frames that correspond to individual images that make up the visual experience of the gaming application 240. During gameplay, a sequence of these video frames is displayed on the GUI of the user device 220 to create motion and interaction, e.g., between player and non-player characters and objects. These video frames are stored in the buffer(s) 250 as gameplay video 254.

The recommendation engine 212 accesses webcam video 252 from the webcam corresponding to the user device 220 and processes these video frames to detect changes in emotional state data for a user of the user device 220. In one implementation, the recommendation engine 212 is configured to execute a Facial Emotion Recognition Model (FER model 262), which is programmed to classify the user's facial expressions detected from the recorded video frames. Each classification generated by the FER model 262, along with a corresponding timestamp is stored in a session file, and session files generated for each user device 220 are stored in the user database 222 (shown as session files 260). In another implementation, if a user device 220 has granted permission for accessing data from one or more corresponding biometric sensors 245, the application system 110 accesses this data through API calls to the biometric sensors 245. In one non-limiting example, this data can include average heart rate data during a gameplay session, along with individual timestamps for instances during a period of time for which the heart rate data has been recorded. In an implementation, when both the webcam video 252 and biometric data is available, the recommendation engine computes the emotional state data using both. Otherwise, the emotional state data is only computed using the webcam video 252. In one example, the biometric data is also added to the session file 260 for the user device 220.

In various implementations, to generate recommendation data for improving gameplay, the recommendation engine 212 is configured to first identify in-game events and correlate each in-game event with corresponding changes in emotional state data using recorded timestamps. In this implementation, to identify in-game events, the recommendation engine 212 is configured to execute a classification model 264 that classifies individual video frames included in the gameplay video 254. In an implementation, the classification model 264 uses the individual video frames as input and outputs a classification of various in-game events identified. When an in-game event is detected, the recommendation engine 212 adds a type of event (e.g., “kills”) and associated timestamp to the corresponding session file 260.

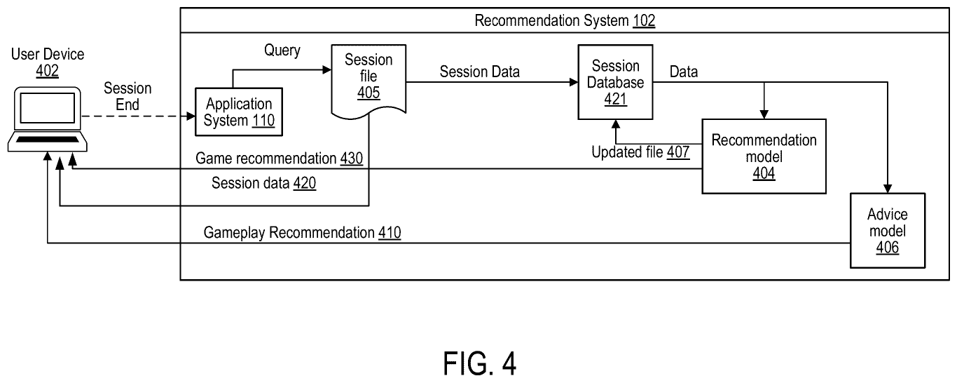

In one implementation, training engine 214 trains a prediction model 266 using the identified in-game events. To train this prediction model 266 for a given user, the training engine 214 uses emotional state data from a session file 260 as input parameters. The prediction model 266 is trained using the input parameters to be able to output predicted in-game events. In one implementation, the training engine 214 uses the previously classified in-game events as ground truth for training the prediction model 266. In machine learning, “ground truth” can refer to actual, real-world data that serve as the standard against which predictions made by a model are evaluated. Once the prediction model 266 is sufficiently trained, the prediction model 266 is then executed to predict different in-game events that would occur as an outcome of changes in emotional state data and biometric data. For instance, for each in-game event, the prediction model 266 is inputted with possible combinations of emotional state data values and a range of biometric data values, and the model 266 outputs predictions of different in-game event occurrences correlated with individual values of emotional state data and/or biometric data values. These predicted outcomes are recorded. These predictions are also included in recommendation data which is presented to a user device 220 when requested, or at an end of a gameplay session executing on the user device 220. This is further described in detail with respect to FIG. 4.

In one implementation, after a gameplay session at a user device 220 ends, the recommendation engine 212 displays recommendation data onto a GUI of the user device 220, including a comprehensive dashboard visually representing emotional state data changes during the gameplay session correlated with various recorded in-game events. Furthermore, the recommendation data further includes outputs from the prediction model 266, i.e., correlations between various possible values of emotional state data (and biometric data) and predicted in-game events that would occur as an outcome of changes in the emotional state data values. This assists users to understand how their emotions impacts gaming performance, and further offers insights to improve future gameplay sessions.

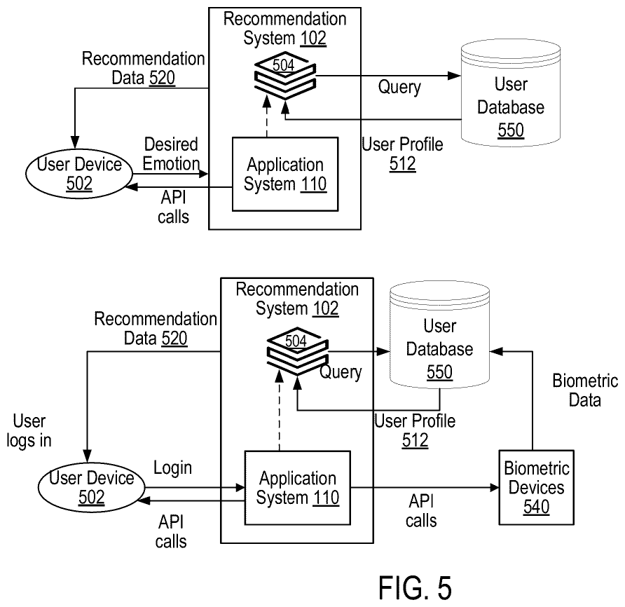

In another implementation, the recommendation engine 212 is further configured to present recommendations of various applications based on currently identified biometric and emotional state data. In this implementation, a login from a user device 220, detected by the application system 110, triggers API calls to biometric sensors 245 and webcam corresponding to the user device 220. The biometric data received from a biometric sensor 245 is then analyzed to detect heart rate data for a user of the user device 220. Further, video frames from the webcam video feed are processed to compute the emotional state data for the user device 220. Based on the current heart rate data (e.g., average heart rate for a given period of time), and emotional state of the user, the recommendation engine 212 generates recommendation data including recommendations for gaming applications for the user that can alter their current emotional state and/or heart rate. In one implementation, the recommendations are generated by executing a recommendation model 268, which is trained on previously generated session files 260. In this implementation, the recommendation model 262 is trained on session files 260 personalized for different users, such that the recommendation model 268 is able to recommend similar gaming applications for similar users. For instance, when it is detected that a player felt calm after playing a particular game (i.e., based on corresponding session file 260), that game is also recommended to the current user, when their emotional state data and biometric data indicates that they are agitated. These implementations are further described in detail with respect to FIG. 5.

In one or more implementations, the recommendation model 262 is designed to improve gameplay for a user. The model 262 is initially trained using historical user data, including previously stored gameplay statistics, patterns, in-game actions, and outcomes from previous gaming sessions. In one implementation, the model 262 employs collaborative filtering, content-based filtering, or a hybrid approach to analyze player behaviors and gameplay features. Collaborative filtering focuses on finding patterns among similar players (e.g., to predict gaming applications for a player that caused a desired emotion in another set of players), while content-based filtering assesses specific gameplay characteristics, such as strategies or actions taken by the user (e.g., based on their emotional state and biometric data), to recommend improvements. During this training phase, the recommendation system 102 associates certain actions with better (or otherwise different) performance by adjusting the model's internal parameters (or weights), which allows the model 262 to make predictions about what strategies might benefit the user based on past data.

Once the model 262 is deployed and starts interacting with active gaming session data, the recommendation system 102 can fine-tune the model 262. For instance, as a user device 220 interacts with a gaming application more, the recommendation system 102 continues to gather data on that individual's evolving gameplay patterns, preferences, and skill levels. Using this data, the system 102 fine-tunes or retrains the model's weights. The recommendation system 102 constantly updates the weights based on new input data, allowing the model 262 to adapt to changes in the user's behavior. For example, if a player demonstrates consistent improvement in a specific area of gameplay, the model 262 is fine-tuned such that it adjusts its weights to prioritize recommendations that reinforce successful strategies. Conversely, if the user struggles with certain recommendations, the model 262 can be fine-tuned to explore alternative strategies that might be more effective. Other implementations are contemplated.

By dynamically recalibrating the weights of the model 262 based on real-time feedback and gameplay performance, the recommendation system 102 causes the model 262 to become more personalized, offering more effective guidance to the user as they progress in their gaming experience. This iterative process of weight fine-tuning ensures that the system 102 is always aligned with the player's current needs, making recommendations that are increasingly tailored to improve their gameplay over time.

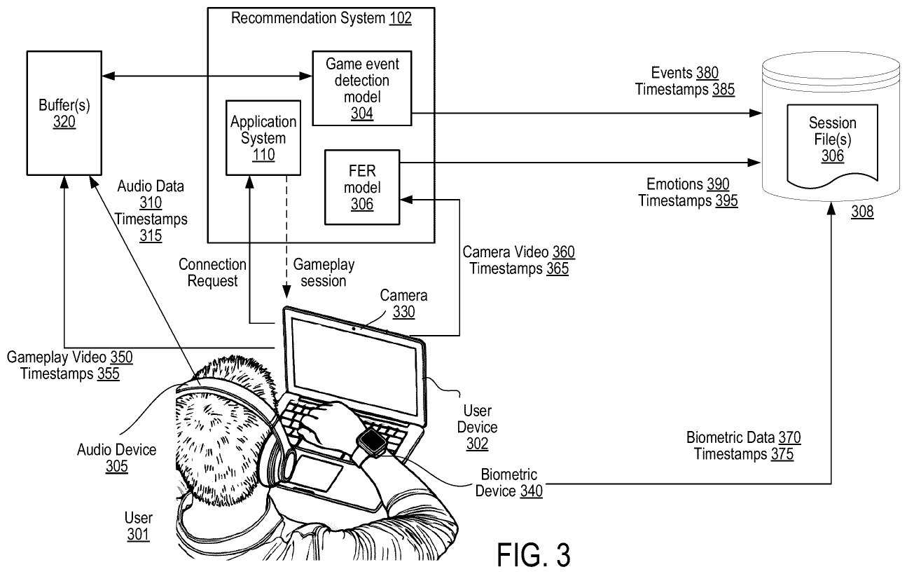

Referring now to FIG. 3, a block diagram illustrating generation of a session file for a user device. As described in the foregoing, recommendation system 102 creates session files for each user device interacting with an application session. Further, each time an application session is complete or terminated, the recommendation system 102 can utilize data from the session file(s) to generate recommendations for the user device.

In the example shown in the figure, a user device 302 logs in to a gameplay session, e.g., by sending a connection request to the application system 110. Responsive to the connection request, the application system 110 enables the user device 302 to access a gaming application. Once the gameplay session starts, the application system 110 is configured to send application programming interface (API) calls to each of a buffer(s) 320, a camera 330, and one or more biometric devices 340. In an implementation, the buffer(s) is configured to store video frames (gameplay video 350) recorded from the execution of the gameplay session onto a GUI of the user device 302. Further, using the API call to the camera 330 (e.g., webcam integrated within the user device 302), the application system 110 causes video feed originating from the camera 330 (camera video 360) to be collected and accessed by the recommendation system 102. Furthermore, biometric data 370, e.g., heart rate data, can be gathered from biometric device 340 (e.g., smartwatch) worn by the user 301. In an implementation, the biometric device 340 can also be connected to the user device 302 (e.g., a smartwatch connected to the user device 302 using Bluetooth®). In one implementation, timestamps associated with each video frame of the gameplay video 350, each video frame of the camera video 360, and biometric data 370 are also collected and correlated. As shown, timestamps 355, 365, and 375, are respectively collected for gameplay video 350, camera video 360, and biometric data 370.

In other implementations, audio data 310 and corresponding timestamps 315 generated by an audio device 305 used by the user 301 are also collected. As shown in the figure, an audio headset is worn by the user 301 while engaging with a gameplay, such that gameplay audio 310 data is recorded in the buffer 320. The gameplay audio 310 data can include audio data originating from the gameplay session as well as audio data generated by the user 301, e.g., collected by means of an microphone corresponding to the audio device 305. In an implementation, collected gameplay video 350 and audio data 310 along with respective timestamps 355 and 315 are utilized by the recommendation system 102 to identify in-game events. In this implementation, the recommendation system 102 accesses data from the buffer(s) 320 and executes a game event detection model 304 using the accessed gameplay video 350, audio data 310 and respective timestamps 355 and 315 as input parameters. The recommendation system 102 uses this data to feed the game event detection model 304, e.g., to classify the buffer 320. In an implementation, the model 304 is a video classification model that uses video and corresponding audio as input and outputs a classification for the video. Further, the model 304 can be fine-tuned for custom data sets so as increase the model 304 accuracy for identifying in-game events. To fine tune the model 304, a range of gameplay video 350 and audio data 310 is used as input and each in-game event outputted is labeled. The model 304 can then be iteratively used with new gameplay video 350 and audio data 310 and previously identified in-game events, thereby enhancing the model's accuracy. The in-game events 380 outputted by the model 304, along with their recorded timestamps 385 are stored in the session file 306 for the user device 302. Each session file(s) 306 generated for the user 301 is stored in user database 308.

The recommendation system 102 further accesses camera video 360 from the camera 330 and processes these video frames to compute emotional state data for the user 301 of the user device 302. In one implementation, the recommendation system 102 is configured to execute a Facial Emotion Recognition Model (FER model 306), which is programmed to classify the facial expressions of the user 301 detected from the video frames from the camera video 360 during different times during the gameplay session (as recorded using the timestamps 365). Each emotion 390 classified by the FER model 306, along with a corresponding timestamp 395 is stored in the session file 306. In one implementation, during the gameplay session, a video is created using a small buffer of video frames from the camera video 360, and fed to the FER model 306, which can include a combination of Convolutional Neural Networks and Long Short-Term Memory networks (i.e., a CNN-LSTM model). This model 306 can take a video as input and output various classifications of emotions 390, such as, ‘Neutral’, ‘Happiness’, ‘Sadness’, ‘Surprise’, ‘Fear’, ‘Disgust’, or ‘Anger’. These emotions 390, correlated with their respective timestamps 395, are stored in the session file 306.

In another implementation, when the user device 302 has granted permission for accessing data from one or more biometric sensors 340, such as smartwatch or smart ring worn by the user 301, the application system 110 accesses this data 370, e.g., including heart rate data and timestamps 375 of individual instances when a reading from a biometric sensor 340 was accessed. In one non-limiting example, this data 370 can include average heart rate data of the user 301 during a gameplay session, along with a period of time for which the heart rate data has been recorded. This data can be used to compute the emotional state of the user 301, whenever available. The biometric data 370 is also added to the session file 306 for the user device 302. The session file(s) 306 are used by the recommendation system 102 to generate recommendation data for enhancing future gameplay sessions for the user. This is discussed with respect to FIG. 4.

FIG. 4 is a block diagram illustrating generation of recommendations for a user interacting with a gaming application session (“gameplay session”). As described in the foregoing, the recommendation system 102 is configured to generate recommendation data for a user of a user device engaged in a gameplay session, so as to assist in enhancing future gameplay sessions of the user.

In the example shown in FIG. 4, a user device 402 (e.g., a gaming console or a mobile device), is currently engaging with a gameplay session. In the implementations described herein, the user device 402 is considered to have enabled receipt of recommendation data from recommendation system 102.

In an implementation, when engaging with the gameplay session, the device 402 is actively participating in an interactive gaming experience, processing inputs (such as touch, button presses, or movements) from the user and rendering real-time responses from the game, such as visuals, sound, and gameplay mechanics. Executing the gameplay session can further involve the user device 402 running game software, sending and receiving data to the application system 110, e.g., to provide a seamless and dynamic user experience during the gameplay session.

The application system 110 is configured to identify the termination of the gameplay session. Termination of the gameplay session can occur when the user device 402 is no longer actively interacting with the game, either by completing the session, quitting, or experiencing a disconnection. At this point, the user device 402 stops processing game-related inputs and outputs, and any ongoing in-game activities are halted.

Once the gameplay session end is indicated by the user device 402 to the application system 110, it sends a query to a session file 405 generated for the user device 402, during the time that the gameplay session was active. In an implementation, the session file includes, without limitation, emotional state data detected for the user of the user device 402. As described earlier, the emotional state data can be computed using webcam video frames as well as collected biometric data. In various implementations, collected data and recommendations can be provided during gameplay (i.e., before termination of a gameplay session). Various such alternatives are possible and contemplated.

The session file further includes different in-game events identified during the gameplay session. In-game events can include, without limitation, completing a level, unlocking a new achievement, gaining experience points, leveling up, receiving an in-game reward, player kills and deaths, player defeating an opponent or getting eliminated etc. Each in-game event (i.e., death of a player character, losing a life in the game, losing/gaining an advantage) is also identified and recorded. As described earlier, the in-game events can be identified using a classification model, that classifies different events, and records these events along with respective timestamps.

The session data retrieved from the session file 405 is stored in a session database 421. In one implementation, to generate advice data, this session data is inputted to an advice model 406, such that the model 406 outputs recommendations that enhances or improves future gameplay sessions for the users. For instance, the advice model 406 is fed an input parameter that states that during the gameplay session, at a first timestamp, a first in-game event occurred in which the user character died. The input parameters further include that the recorded emotional state data value is “happy,” and the recorded heart rate value is within range (e.g., between 75-105 BPM). The advice model 406 is further fed with other emotional state data values, e.g., ‘Neutral’, Happy’, ‘Sadness’, ‘Surprise’, ‘Fear’, ‘Disgust’, or ‘Anger’ and a range of different heart rates. Based on these inputs, the advice model 406 predicts different events that would occur as an outcome of changes in emotional state data values and heart rate ranges. For instance, the advice model 406 predicts that the user would have gotten a kill instead of dying had their heart rate been within a particular range, and their emotional state data value was “angry”. The advice model 406 outputs such gameplay advice for all captured in-game events in the gameplay session.

In one implementation, each prediction generated by the model 406, along with its corresponding timestamp, would be added to an updated session file 407. Further, in order to provide safe advice, the system 102 monitors the user's heart rate at rest before starting the gameplay session (assuming that access to the biometric data is available). This serves both to get a baseline comparison for future recommendations and detect heart conditions such as Tachycardia or Bradycardia. In case the user is detected as having a heart condition, this is also indicated in the advice data. As shown, the predictions outputted by the advice model 406 are sent as gameplay recommendation 410 to the user device 402. A summary of the session data 420 is also transmitted to the user device 402.

In an implementation, a recommendation model further uses the session data 404 to generate recommendations for other gaming applications based on the user's emotional state data and biometric data. To recommend other games to the user device 402, user's emotional state data is collected both before starting the gameplay session and during the gameplay session. This data is stored in the session file 405. In one implementation, the recommendation model 404 uses K-Means clustering to identify which emotion the gameplay session produces for the user. The recommendation model 404 then identifies gaming applications that are currently installed on the user device 402, e.g., by searching game folders and recommending gaming application(s) that produces a given emotion. The recommended gaming applications are then presented to the user device 402, as shown by game recommendation 430.

In one or more implementations, the system 102 utilizes data analysis, leveraging inputs such as facial expressions, audio data, and/or interaction behaviors to assess the user's current emotional state. Further, advanced machine learning models, such as recommendation model 404, processes this data to align an identified mood of the user with a database of apps tagged according to their relevance to different emotional states. In an example, if a user is identified as being stressed, the system 102 may suggest calming or strategy-based games to the user. Conversely, if the user shows signs of excitement or a high-energy mood, action-oriented or competitive games might be recommended. The system 102, in one implementation, continuously updates the recommendations as the user's mood changes, thereby ensuring a tailored and engaging user experience. These and other implementations of recommending gaming applications are described with respect to FIG. 5.

Turning now to FIG. 5, a block diagram illustrating generation of gameplay recommendations for a user are described. As described in the foregoing, recommendation system 102 is configured to generate recommendations of gaming applications for a user device, based on emotional state data and biometric data corresponding to the user.

The top half of the figure describes an implementation when the recommendations are generated based on a current emotion or desired emotion for the user, while the second half of the figure describes an implementation wherein recommendations are generated based on the computed emotional state data and the biometric data (e.g., heart rate data) corresponding to the user. As shown in the first example, a user device 502 requests a gaming application recommendation from the recommendation system 102. In an implementation, the user can either manually enter a desired emotion for which recommendation is sought, or provide permission to the application system 110 to access webcam data (through API calls to the user device 502) to compute current emotional state data (e.g., using a FER model). In either case, the recommendation system is configured to execute a game recommendation model 504 with the desired (or detected) emotion. The game recommendation model queries a user database 550 for previously stored user profiles corresponding to the user device 502, and a profile 512 is inputted to the game recommendation model 504, as shown. This profile 512 is used by the game recommendation mode 504 as input parameter to output one or more gaming applications as recommendations. In one implementation, the one or more gaming applications are suggested that can effectively cause the desired emotion for the user. In another implementation, the recommendation model 504 can recommend a gaming application for a user, when another user with a similar profile felt the desired emotion after engaging with the gaming application. The recommendations are sent to the system 502 as recommendation data 520.

As shown in the second example, the user device 502 logs in to the application system 110. In this example, the application system retrieves webcam video feed from the user device 502, as well as biometric data from the biometric devices 540. In one implementation, both the webcam video feed and the biometric data is accessed by sending API calls to a webcam (not shown) corresponding to the user device 502, and to the biometric devices 540, respectively. The biometric data and the webcam video feed are used to compute the emotional state data and the computed data is stored in the user database 550. Further, a user profile 512 is updated with the computed data.

For generating recommendations, the recommendation system 102 executes the recommendation model 504. The recommendation model 504 uses the user profile 512 as input, wherein the profile 512 contains the current emotional state data, biometric data, and previously recommended gaming applications. Based on processing the input, the recommendation model 504 outputs recommendation data that includes one or more gaming application suggested to the user based on their current emotional state data, mood, and/or biometric data. The recommendation data includes gaming application(s) that can effectively improve the user's emotional state. For instance, the recommendation model 504 can recommend a particular game if a previous user with a similar profile felt calm after playing a particular game, and it is detected that the user is feeling down. Other implementations are possible and are contemplated.

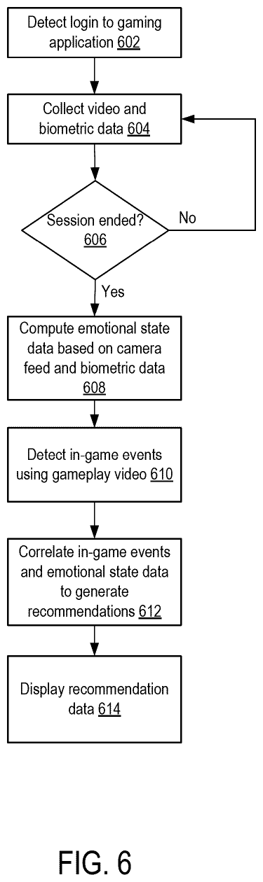

FIG. 6 illustrates a method for generating gameplay recommendations. In one implementation, a recommendation system (e.g., system 102) detects a user device login for a gaming application (block 602). The recommendation system begins collecting video data and biometric data for the user device (block 604). In one implementation, the video data includes gameplay video data and camera feed from a webcam associated with the user device. Further, biometric data is accessed from one or more biometric devices (e.g., heart rate sensors, smartwatches etc.) connected to the user device.

The recommendation system is configured to detect whether the session is terminated by the user device (conditional block 606). If the session is still active (conditional block 606, “no” leg), the method continues to block 604. When the session is terminated (conditional block 606, “yes” leg), the recommendation system computes emotional state data for a user of the user device based on the camera feed and the biometric data (block 608). In an implementation, the camera feed is inputted to a Facial Emotion Recognition (FER) model and emotional state data is outputted. Further, the emotional state data and the biometric data with their corresponding timestamps are recorded in a database.

The recommendation system is further configured to detect in-game events based on the gameplay video (block 610). In an implementation, the gameplay video is inputted to a classification model that outputs classified in-game events, with each in-game event labeled with the identified class. The in-game events along with respective timestamps, i.e., individual instances during the gameplay session when these in-game events occurred, are stored in a database.

Based on the detected in-game events correlated with the computed emotional state data, the recommendation system generates recommendation data (block 612). The recommendation data at least includes different values of emotional state data and biometric data that would cause a different in-game event than originally identified (i.e., a predicted change in an in-game event that would occur as an outcome of changes in emotional state data). The recommendation data further includes recommendations of other gaming applications that are identified based on the current emotional state of the user and/or desired emotional state of the user. The generated recommendation data is presented to the user device (block 614).

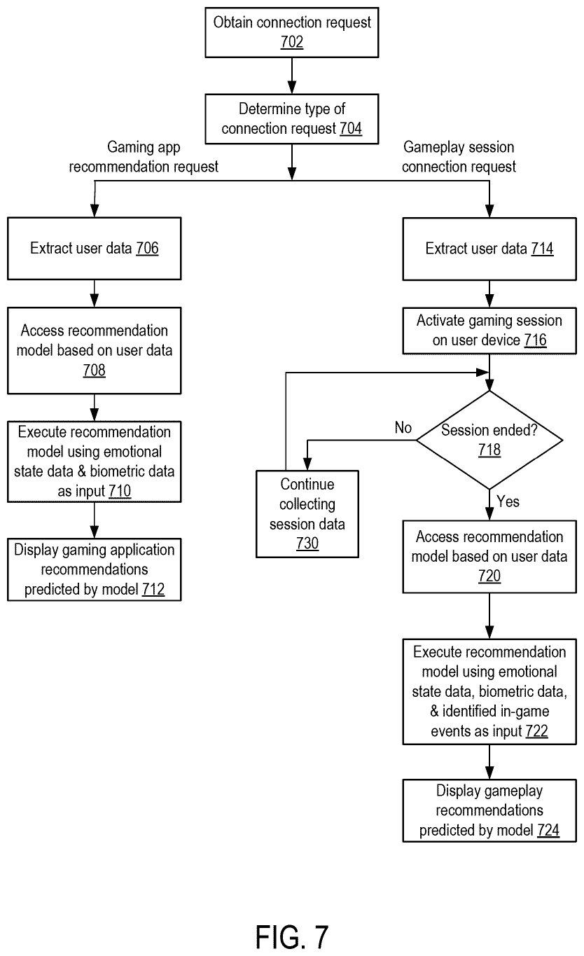

FIG. 7 illustrates another method for generating gameplay recommendations. In one or more implementations, a processing circuitry is configured to obtain a connection request from a user device (block 702). Responsive to the request, the processing circuitry is configured to determine the type of connection request received from the user device (block 706). The type of connection requests can include a gaming application recommendation request and a gameplay session connection request.

In case of a gaming application recommendation request, the processing circuitry is configured to extract user data from a user database (block 706). The user data can include information such as but not limiting to, username, password, registration information, application usage history, application ownership, preferences, and other settings. Next, the processing circuitry accesses a recommendation model based on the extracted user data (block 708). In an implementation, the recommendation model is trained based on historical user data (as weights) so as to cause the recommendation model to generate recommendations as predicted outputs. The processing circuitry executes the recommendation model using current emotional state data and biometric data as input parameters (block 710). The recommendation model outputs one or more gaming application recommendations based on the current emotional state detected for the user (or a desired emotional state requested by the user). The recommendations are then displayed onto a graphical user interface (GUI) of user device (block 712).

In one or more implementations, if the user device is connecting with the system for the first time, the processing circuitry can provide an option to register. A user of the user device can enter required registration information and register the user device with the recommendation system. In such implementations, identification of whether a user device is a registered or unregistered device may be made at least based on user data stored at user database.

Referring again to block 704, when the connection request is a request to connect to a gameplay session, the processing circuitry again extracts user data (block 714). Based on the extracted user data, the processing circuitry activates the gaming session on the user device (block 716). The processing circuitry then determines whether the active session is terminated by the user (conditional block 718). Till the time the session is not terminated (conditional block 718, “no” leg), the processing circuitry keeps collecting session data from the user device (block 730). The session data includes in-game events, corresponding timestamps, emotional state data of the user, and biometric data of the user, collected during a time period the user device is actively engaging with the application session. Once the session ends (conditional block 718, “yes” leg), the processing circuitry accesses the recommendation model based on the user data and collected session data (block 720).

The circuitry executes the recommendation model by inputting in-game events correlated with corresponding emotional state data and biometric data as input parameters to the recommendation model (block 722). The recommendation model outputs recommendation data, wherein this data at least includes a suggestion for the user to improve their gameplay. In one example, the recommendation data is indicative of different in-game event outcome predictions. For instance, the recommendation data can indicate to the user that they would have gotten a kill instead of dying had their heart rate been high and their emotional state was angry. These recommendations can be captured for all in-game events in the session. Further, the processing circuitry is configured to organize the recommendation data into a graph. This recommendation data is displayed at a GUI of the user device (block 724).

It should be emphasized that the above-described implementations are only non-limiting examples of implementations. Numerous variations and modifications will become apparent to those skilled in the art once the above disclosure is fully appreciated. It is intended that the following claims be interpreted to embrace all such variations and modifications.

What is claimed is

1. A system comprising:
processing circuitry configured to:
generate correlation data that indicates a correlation of emotional state data corresponding to a user of a user device with a first application event of a first application executing on the user device; and
generate, based at least in part on the correlation data, recommendation data indicative of a change in the emotional state data that would cause a second application event different from the first application event.

2. The system as claimed in claim 1, wherein the recommendation data is further indicative of one or more predicted application events that would result responsive to one or more changes in the emotional state data.

3. The system as claimed in claim 1, wherein the processing circuitry is configured to:
input, to a machine learning model trained to correlate emotional state data with application events, the change in the emotional state data; and
predict, using the machine learning model, the second application event.

4. The system as claimed in claim 3, wherein the processing circuitry is configured to use the emotional state data correlated with the first application event as training data for the machine learning model.

5. The system as claimed in claim 1, wherein the processing circuitry is configured to compute the emotional state data at least in part based on one or more of biometric data corresponding to the user or video data.

6. The system as claimed in claim 1, wherein the processing circuitry is configured to generate the recommendation data for display to the user, responsive to termination of an active application session of the first application.

7. The system as claimed in claim 1, wherein the recommendation data is further indicative of a second application selected corresponding to the emotional state data.

8. A method comprising:
Generating, by circuitry, correlation data that indicates a correlation of emotional state data corresponding to a user of a user device with a first application event of a first application executing on the user device; and
generating, by the circuitry, based at least in part on the correlation data, recommendation data indicative of a change in the emotional state data that would cause a second application event different from the first application event.

9. The method as claimed in claim 8, wherein the recommendation data is further indicative of one or more predicted application events that would result responsive to one or more changes in the emotional state data.

10. The method as claimed in claim 8, further comprising:
inputting, by the circuitry, to a machine learning model trained to correlate emotional state data with application events, the change in the emotional state data; and
predicting, by the circuitry using the machine learning model, the second application event.

11. The method as claimed in claim 10, further comprising using, by the circuitry, the emotional state data correlated with the first application event as training data for the machine learning model.

12. The method as claimed in claim 8, further comprising computing, by the circuitry, the emotional state data at least in part based on biometric data corresponding to the user.

13. The method as claimed in claim 8, further comprising generating, by the circuitry, the recommendation data for display to the user, responsive to termination of an active application session of the first application.

14. The method as claimed in claim 8, wherein the recommendation data is further indicative of a second application selected corresponding to the emotional state data.

15. A recommendation system comprising:
a memory; and
at least one processor configured to:
compute emotional state data corresponding to a user of a user device;
correlate the emotional state data with an application event of a first application executing on the user device;
generate, based at least in part on the correlation, recommendation data indicative of a change in the emotional state data that would cause a second application event different from the first application event; and
cause the recommendation data to be displayed on a graphical user interface (GUI) of the user device.

16. The recommendation system as claimed in claim 15, wherein the recommendation data is further indicative of one or more predicted application events that would result responsive to one or more changes in the computed emotional state data.

17. The recommendation system as claimed in claim 15, wherein the at least one processor is configured to:
input, to a machine learning model trained to correlate emotional state data with application events, the change in the emotional state data; and
predict, using the machine learning model, the second application event.

18. The recommendation system as claimed in claim 17, wherein the at least one processor is configured to use the emotional state data correlated with the first application event as training data for the machine learning model.

19. The recommendation system as claimed in claim 15, wherein the at least one processor is configured to display the recommendation data on the GUI of the user device, responsive to termination of an active application session of the first application.

20. The recommendation system as claimed in claim 15, wherein the recommendation data is further indicative of a second application selected corresponding to the computed emotional state data.
